(19) United States
(12) Patent Application Publication
(10) Pub. No.: US 2015/0071980 A1
(43) Pub. Date: Mar. 12, 2015

(54) FORMATION OF NANOSIZED METAL PARTICLES ON A TITANATE CARRIER

(71) Applicant: Savannah River Nuclear Solutions, LLC, Aiken, SC (US)

(72) Inventors: David T. Hobbs, North Augusta, SC (US); Kathryn M.L. Taylor-Pashow, Aiken, SC (US); Mark C. Elvington, Fort Worth, TX (US)

(73) Assignee: Savannah River Nuclear Solutions, LLC, Aiken, SC (US)

(21) Appl. No.: 14/020,472

(22) Filed: Sep. 6, 2013

(51) Int. Cl. C23C 18/44; H01M 4/62; C23C 18/18; A61K 9/14; H01G 9/20; A61L 27/30; A61L 26/00; H01M 4/92; H01M 4/88; B01J 23/58; A61K 33/24

(52) U.S. Cl. CPC C23C 18/44; B01J 23/58; H01M 4/624; C23C 18/1882; A61K 9/14; A61K 33/24; A61L 27/306; A61L 26/0095; A61L 26/0066; H01M 4/925; H01M 4/8842; H01G 9/20
USPC 424/421; 502/350; 502/344; 502/5; 502/339; 427/343; 427/553; 424/490; 424/649; 106/35; 428/404

(57) ABSTRACT

Methods directed to the synthesis of metal nanoparticles are described. A formation process can be carried out at ambient temperature and pressure and includes the deposition of metal ions on a titanate carrier according to a chemical deposition process followed by exposure of the metal ions to a reducing agent. Upon the exposure, nanoparticles of the reduced metal are formed that are adhered to the titanate carrier.

### STATEMENT AS TO RIGHTS TO INVENTIONS MADE UNDER FEDERALLY SPONSORED RESEARCH

This invention was made with Government support under Contract No. DE-AC09-08SR22470 awarded by the United States Department of Energy and under Grant #1R01DE021373-01 awarded by the National Institute of Health. The Government has certain rights in the invention.

### BACKGROUND

Metal nanoparticles have found a wide variety of uses such as catalysts, in imaging, and in medical applications. For instance, gold nanoparticles and composite materials including gold nanoparticles have found use in photovoltaic applications and surface enhanced Raman scattering (SERS). Gold in the form of nanoparticles and ions is also used in medical applications such as imaging, drug delivery, and disease treatment and shows promise for use as a biocide. For instance, in vitro tests of monosodium titanate supports carrying Au(III) ions indicate suppression of the growth of cancer and bacterial cells.

Unfortunately, nanosized materials are very difficult to work with. For instance, gold nanoparticles are very mobile and possess large surface energy and, therefore, particles tend to coagulate easily. In fact, it has been difficult to prevent such coagulation from occurring. Moreover, the activity of many metals tends to fall off as the particle size increases. Therefore, the development of methods to deposit and immobilize nanosized metal particles on a carrier in a uniformly dispersed state has been of interest, particularly for precious metals such as gold, platinum, silver, and so forth.

Supported precious metal nanoparticles are typically prepared by one of three processes: a) co-precipitation, b) deposition/precipitation, or c) impregnation processes. In co-precipitation processes, soluble precursors of both the support and the precious metal are precipitated from solution together (e.g., by adjusting the pH through addition of a base such as sodium hydroxide to form hydroxide precipitates) followed by drying, calcination, and reduction of the precious metal precipitate to metallic form. Deposition-precipitation methods involve precipitation (e.g., by adjusting pH) of a salt or hydroxide of the precious metal in the presence of a suspension of the support, followed by drying, calcination and reduction, typically high-temperature gaseous reduction, to form the metallic particles. The final preparation process type, impregnation, is achieved by wetting dry support particles with a solution of a solubilized precious metal such that the precious metal solution impregnates the pores of the support. Following impregnation, the support is dried, causing the precious metal salt to precipitate in the pores. The support is then calcined and exposed to a reducing gas to form metallic particles within the pores. Other less common procedures such as the use of colloids, grafting and vapor deposition have met with varying degrees of success.

Unfortunately, known methods suffer from serious difficulties, reproducibility being one of the primary problems. The issues surrounding the difficulties include difficulty in controlling particle size, poisoning of catalyst by ions such as chlorine, loss of active metal in the pores of the carrier and/or in the formation solution, inactivation of certain catalytic sites by thermal treatment, the lack of control of metal oxidation state, and the inhomogeneous nature of metallic solutions upon the addition of a base (e.g., metal nanoparticles may be reduced in solution prior to adhering to the support). Moreover, known methods are often complicated and expensive due to, e.g., the necessity of thermal treatments to activate the catalysts and the requirement for accurate control of deposition conditions over a long period of time. In addition, in an attempt to improve efficiency, additional steps have been instituted to recover the metals from the deposition solution, leading to increased complication of the processes.

In view of the above, what is needed in the art is a simple, efficient, and reproducible method of forming nanosized metal particles on a support and the products formed thereby that could be suitable for use in a variety of applications, and in one embodiment for biological applications.

Moreover, the particle size of the support would be expected to significantly affect the nature of the interaction between the nanosized metal carried on the support and the target, e.g., a biological target such as cancer or bacterial cells. Accordingly, what are also needed in the art are methods for the synthesis of nanosized metal particles on nanosized supports. Such nanosized materials could be utilized to enhance ion exchange kinetics and effective capacity in metal ion separation, enhance photochemical properties, as well as to facilitate metal delivery and cellular uptake from the delivery platform.

### SUMMARY

Aspects and advantages of the invention will be set forth in part in the following description, or may be obvious from the description, or may be learned through practice of the invention.

According to one embodiment, disclosed is a method for forming metal nanoparticles. For example, the method can include depositing metal ions on a titanate carrier, the metal ions being at a known oxidation state. Following the deposition of the metal ions, the metal ions held on the carrier can be exposed to a reducing agent. Upon the exposure of the metal ions and the carrier to the reducing agent, nanoparticles of the metal can be formed and the metal can be reduced as compared to the oxidation state of the metal ions.

Also disclosed are composite materials as may be formed according to the methods. The composite can include metal nanoparticles adhered to a titanate carrier. Beneficially, in one embodiment the formed composite can be free of organic surfactants.

### BRIEF DESCRIPTION OF THE FIGURES

A full and enabling disclosure of the present invention, including the best mode thereof, directed to one of ordinary skill in the art, is set forth in the specification, which makes reference to the appended figure, in which:

FIG. 1A and FIG. 1B provide transmission electron microscopy (TEM) images for nanosized monosodium titanate.

FIG. 2 illustrates Au(III)-nanosized monosodium titanate composite held in a water/ethanol solutions over 72 hours as the Au(III) is reduced to Au(0) and the gold nanoparticles form including two images at time zero (FIG. 2A, FIG. 2B), and images at 4 hours (FIG. 2C), 6 hours (FIG. 2D), 24 hours (FIG. 2E) and 72 hours (FIG. 2F).

FIG. 3A and FIG. 3B illustrate gold nanoparticles formed on nanosized monosodium titanate carrier particles.

FIG. 4 illustrates Au(III)-micron-sized monosodium titanate composite held in a water/ethanol solution over one week as the Au(III) is reduced to Au(0) and the gold nanoparticles form including images at time zero (FIG. 4A), 1 hour (FIG. 4B), 2 hours (FIG. 4C), 3 hours (FIG. 4D), 4 hours (FIG. 4E), 6 hours (FIG. 4F), 8 hours (FIG. 4G), and 1 week (FIG. 4H).

FIG. 5 presents transmission electron microscopy (TEM) images of gold nanoparticles produced on micron-sized monosodium titanate. Some of the samples were stored in the dark during the nanoparticle formation (FIG. 5A, FIG. 5B, FIG. 5C) and some were stored in the light during the nanoparticle formation (FIG. 5D, FIG. 5E, FIG. 5F).

### DETAILED DESCRIPTION

It is to be understood by one of ordinary skill in the art that the present discussion is a description of exemplary embodiments only, and is not intended as limiting the broader aspects of the present disclosure. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

In general, disclosed herein are methods directed to the synthesis of metal nanoparticles. More specifically, the metal nanoparticles can be formed on a titanate carrier. As utilized herein, the term ‘titanate carrier’ generally refers to a particle that includes titanate as a component of the particle, for example a micron-sized or nanosized monosodium titanate particle or a sodium titanium oxide nanotube. Beneficially, the formation process can be carried out at ambient temperature and pressure and the formed composites including the metal nanoparticles adhered to the titanate carrier can be free of organic surfactants. As such, the composites can provide the desired activity of the metal (e.g., catalytic activity, electrical conductivity, etc.) without interference of extraneous materials such as organic polymers that may detrimentally affect the desired activity.

The formation method generally includes deposition of metal ions on the carrier according to a chemical deposition process followed by reduction of the metal ions to form the metal nanoparticles adhered to the carrier. The metal of the metal nanoparticles is not particularly limited, provided a cation of the metal can be held in solution and can exchange with sodium in an ion exchange chemical deposition process. By way of example, the metal can be a transition metal including, without limitation, chromium, manganese, iron, cobalt, nickel, and copper. In one embodiment, the metal can be a transition metal of the platinum group such as platinum, palladium, rhodium, ruthenium, silver, or gold.

According to the formation process an ion exchange process is carried out between a cation of the metal and sodium of the titanate carrier. More specifically, an aqueous solution of the metal ion can be combined with a suspension of the titanate carrier for a period of time under agitation to encourage ion exchange between the metal ions and sodium ions of the carrier.

In one embodiment, the titanate carrier can be micron-sized or nanosized monosodium titanate. Monosodium titanate is a white, inorganic, and amorphous sodium titanate that can have the general composition of HNaTi₂O₅.xH₂O where x is about 2 to about 4. The materials can exhibit high selectivity for sorbing various metallic ions over a wide pH range extending from about pH 2 to more than pH 14.

Nanosized monosodium titanate can be formed according to a sol-gel process that includes the precipitation of the monosodium titanate in a semi-particulate/semi-gel-like state from a reactant mixture followed by a heating step to complete the particulate formation of the nano-sized product. The reactant mixture is formed by the combination of multiple different solutions. Beneficially, the formation process can take place at atmospheric pressure and can be carried out by combining all of the reactants in a single step; i.e., there is no need for an initial seed formation step followed by a second reactant addition step so as to grow the product particles, as is required in the formation of micro-sized monosodium titanates.

One of the solutions for forming the nanosized monosodium titanate can include a titanium alkoxide, a sodium alkoxide, and an alcohol.

Examples of titanium alkoxide compounds that can be used include, without limitation, titanium methoxide, titanium ethoxide, titanium isopropoxide, titanium propoxide, and titanium butoxide. Equivalent names for titanium isopropoxide (C₁₂H₂₈O₄Ti) include: tetraisopropyl orthotitanate, titanium tetraisopropylate; tetraisopropyl titanate; isopropyl titanate; titanium isopropoxide; titanium(IV) i-propoxide; tetraisopropoxytitanium(IV), tetraisopropyl orthotitanate; titanium iso-propylate; and orthotitanic acid tetraisopropyl ester.

Examples of sodium alkoxide compounds that can be used include, without limitation, sodium methoxide (NaOCH₃) (alternatively referred to as sodium methylate), sodium ethoxide, sodium propoxide, and sodium butoxide and mixtures thereof.

Alcohols that can be utilized as solvent include alcohols that are miscible in water such as, without limitation, isopropyl alcohol, methanol, ethanol, propanol, butanol, isopropanol, and mixtures thereof.

As previously mentioned, the reactants can be utilized in lower concentrations than has been utilized in the past for formation of monosodium titanate. For example, a solution of the titanium alkoxide and sodium alkoxide reactants in an alcohol solution having respective concentrations of about 800 millimolar (mmolar) and about 400 mmolar or less. In some embodiments, this solution can include the titanium alkoxide in a concentration of from about 0.30 mmolar to about 0.61 mmolar and the sodium alkoxide in a concentration of from about 0.15 mmolar to about 0.30 mmolar.

A second reactant solution of the formation process includes water and an alcohol. The alcohol can be the same or different as the alcohol utilized in forming the first reactant solution. The water can generally be ultrapure water (though this is not a requirement of the formation process) and can be provided in this solution at a concentration of about 2.0 molar or less, or between about 1.2 molar and about 1.6 molar in some embodiments.

A third reactant solution of the formation process includes an alcohol (either the same or different as is utilized in forming the other solutions) and a non-ionic surfactant. In general, the non-ionic surfactant can be provided in this solution at a concentration of from about 0.01 moles per mole of the titanium alkoxide component to about 1.2 moles per mole of the titanium alkoxide component, for instance from about 0.05 moles per mole of the titanium alkoxide component to about 0.5 moles per mole of the titanium alkoxide component, or from about 0.1 moles per mole of the titanium alkoxide component to about 0.15 moles per mole of the titanium alkoxide component.

Examples of nonionic surfactants include, but are not limited to, polyethoxylates; polyethoxylated alkylphenols; fatty acid ethanol amides; complex polymers of ethylene oxide, propylene oxide, and alcohols; and polysiloxane polyethers. In one embodiment, the nonionic surfactant can be an aryl alcohol ethoxylate such as those available from Union Carbide under the trade name Triton®. Non-limiting examples of a particular nonionic surfactants include Triton® X-100 that includes an octyl phenol ethoxylate having approximately 9.5 ethylene oxide units, and Triton® X-165 that includes an octyl phenol ethoxylate having approximately 16 ethylene oxide units. In particular, the particle size of the nanosized monosodium titanate appears to be independent of the number of ethylene oxide units of an aryl alcohol ethoxylate surfactant.

Other suitable non-ionic surfactants can include alkyl alcohol ethyoxylates such as a linear alkyl alcohol ethoxylate. A linear alkyl alcohol ethoxylate can include an aliphatic ethoxylate having from about two to twenty-five carbons in the alkyl chain such as from about five to about eighteen carbons in the alkyl chain. In addition, the alkyl alcohol ethoxylate can include from about four to about twelve ethylene oxide units. Exemplary commercially available linear alkyl ethoxylates are available from Sigma-Aldrich under the Brij® designation such as Brij® 52. Another alkyl alcohol ethyoxylate that can be utilized is 2,3,7,9-tetramethyl-5-decyne-4,7-diol ethoxylate available under the trade name Aldrich-461180 from Sigma-Aldrich.

Additional non-ionic surfactants as may be utilized include phosphate surfactants such as polyethylene glycol phosphate (e.g., Merpol® A available from Sigma Aldrich), polyoxyethylene sorbital monolaurate available from Sigma-Aldrich under the name Tween® 20, and linear polymers of ethylene oxide comprising a perfluorinated alkyl chain at one terminus and a hydroxyl group or alkyl group at the other terminus, one example of which include the Zonyl® line of surfactants that are commercially available from Sigma Aldrich, Zonyl® FS300 being one example thereof.

In one embodiment, the solutions can be combined in a single mixing step in which the first solution including the titanium alkoxide, the sodium alkoxide, and alcohol and the second solution including water and alcohol are simultaneously added to the third solution including alcohol and the surfactant. The first and second solutions can generally be added relatively slowly, for instance at a rate about 1.0 cm³/min or less, for instance about 0.5 cm³/min or less.

Following formation, the reaction mixture can be sealed, stirred and then heated. For instance, following stirring for a period of time (e.g., about 24 hours), the reaction mixture can be heated to a temperature corresponding to the boiling point of the alcohol and water azeotrope. For isopropanol the azeotrope boils at from about 80° C. to about 82° C. As the alcohol evaporates during the heating step, water can be added to the reaction mixture. The heating can continue until most of the alcohol has evaporated, for example from about 45 minutes to about 90 minutes, following which the container holding the mixture can be purged, for instance with nitrogen, while the mixture is still at the increased temperature.

The resulting mixture can include the formed nanosized monosodium titanate in an aqueous-based slurry. The particulate can be held in the slurry for storage or use or can be separated, washed, and optionally dried according to any suitable process as is known in the art. By way of example, the slurry can be placed on a filter unit connected to a vacuum line. A vacuum can be pulled from beneath the filter, which pulls the supernatant liquid through the filter. The liquid, referred to as filtrate, can typically be collected and discarded. The solids collect on the surface of the filter and are typically referred to as a filter cake. Finally, water (or other solvents could be used) can then be added to the top of the filter cake and allowed to flow through the solids to displace any remaining alcohol and surfactant that remain with the solids. This water-washing step may be repeated several times, so that liquid remaining in the filter cake becomes essentially that of the washing fluid (e.g., water), with essentially no alcohol or surfactant remaining. In one embodiment, the filter cake can then be dried, for instance in air at room temperature, under vacuum at increased temperature, or some combination thereof, to provide a dried particulate product.

The nanosized monosodium titanate particulate formed according to the disclosed methods can exhibit spherical-shaped particle morphology with a monodisperse distribution of particle diameters. For instance, the maximum particle cross-sectional dimension can be about 1000 nm or less, about 500 nm or less, or about 300 nm or less in some embodiments. For example, the maximum particle cross sectional dimension can be in the range from 100 to 150 nm.

The BET surface area and isoelectric point of the nanosized materials can be more than an order of magnitude higher and a pH unit lower, respectively, than that measured for larger micro-sized MST. For example, the BET surface area can be about 200 m²g⁻¹ or greater. In some embodiments the BET surface area can be from about 200 m² g⁻¹ to about 350 m² g⁻¹, for instance 285 m² g⁻¹. The isoelectric point can be from about 3.1 pH units to about 3.5 pH units in some embodiments, for instance 3.34 pH units in one embodiment.

The titanate carrier is not limited to nanosized titanate materials. In one embodiment, a micron-sized titanate carrier can be utilized. A micron-sized titanate carrier can generally have a maximum cross-sectional area of from about 1000 nanometer to about 1 millimeter. In one embodiment, micron-sized monosodium titanate can be utilized. Micron-sized monosodium titanate can be obtained on the retail market (for instance Optima 00-QAB-417). Alternatively, micron-sized monosodium titanate can be produced according to standard methodology as fine powders, with particle sizes ranging from a few to several hundred microns using either sol-gel or hydrothermal synthetic techniques. By way of example, in one embodiment, micron-sized monosodium titanate can be prepared by a sol-gel method in which tetraisopropoxytitanium(IV) (TIPT), sodium methoxide and water are combined and reacted in isopropanol to form seed particles of monosodium titanate. Micron sized particles can then be grown by controlled addition of additional quantities of the reagents resulting in a particle morphology that features an amorphous core and an outer fibrous region.

The titanate carrier is not limited to monosodium titanate. For example, in one embodiment, the titanate carrier can be peroxo-titanate, which can be formed by treatment of the micron-sized or nanosized monosodium titanate with a peroxide to convert the monosodium titanate to a peroxo-titanate form, which has been shown to improve the sorption capabilities of the materials. When utilized, a peroxo-titanate carrier may retard the subsequent reduction of the metal and the formation of the metal nanoparticles. Accordingly, a peroxo-titanate carrier may be utilized in those embodiments in which it is preferred that the nanoparticle formation and metal reduction is delayed, for instance following a period of storage and/or shipment of the metal ion/carrier composite.

The general formula of peroxo-titanate as may be utilized as a carrier for the metal nanoparticles is HᵥNa_{w}Ti₂O₅.(xH₂O)[yH₂O] where v+w=2 and z=0 to 2. For peroxo-titanates synthesized under neutral or basic conditions, v≈w≈1. For acid-treated peroxo-titanates, v>w. The species in the square brackets is peroxide, which is most likely coordinated to the titanium and may be present as O₂², HO₂⁻, or H₂O₂ (see Nyman, et al., Chem. Mater. 2006, 18, 6425-6435).

Peroxide treatment of the monosodium titanate can be carried out in one embodiment according to methods as described in U.S. Pat. No. 7,494,640 to Nyman, et al., which is incorporated herein by reference. For example, a solution of hydrogen peroxide (e.g., about 30 wt. % hydrogen peroxide) can be added dropwise to a suspension of monosodium titanate. The reaction mixture can be stirred for a period of time (e.g., about 24 hours) at ambient temperature. Upon treatment, the color of the monosodium titanate will change from white to yellow. The yellow color is due to the η²-bound protonated hydroperoxo-titanium ligand-to-metal-charge-transfer absorption at 385 nm. Peroxide treatment of the monosodium titanate can be carried out without alteration of the particle size or morphology of the particulates.

In another embodiment, the titanate carrier can include sodium titanium oxide nanotubes. Sodium titanium oxide nanotubes can be formed according to methods as are generally known in the art (see, e.g., Chen, W.; Guo, X.; Zhang, S.; Jin.Z. (2007) TEM study on the formation mechanism of sodium titanate nanotubes J. Nanoparticle Res. 9, 1173-1180; Menga, X.; Wanga, D.; Liva, J.; Zhang, S. (2004) Preparation and characterization of sodium titanate nanowires from brookite nanocrystallites Mat. Res. Bul. 39 2163-2170; Yada, M.; Goto, Y.; Uota, M.; Torikai, T.; Watari, T. (2006) Layered sodium titanate nanofiber and microsphere synthesized from peroxotitanic acid solution J. Eur. Ceram. Soc. 26, 673-678). In one embodiment, sodium titanium oxide nanotubes can be formed via hydrothermal processes in which titanium dioxide is reacted with excess sodium hydroxide at elevated temperature and pressure. The formation of sodium titanate nanotubes in the form of Na₂Ti₂O₄(OH)₂ ensues by self-assembly of the dissolved intermediate of titanium dioxide and sodium hydroxide.

Following the ion exchange process, the titanate carrier can include the metal ions. The metal ions will be in a predetermined oxidation state. For instance, an ion exchange process carried out with a gold solution can deposit the gold ions on the titanate carrier in the Au(III) oxidation state. The particular oxidation state of the metal ions is not of critical importance, provided that the metal ions can exchange with the sodium ions of the titanate carrier and be coordinated with the nanosized titanate carrier.

To form the metal nanoparticles, the titanate carrier including the metal ions can be exposed to a reducing agent. While not wishing to be bound to any particular theory, the nanoparticles are believed to form following transport of the oxidized ion to the reactive surface followed by reduction of the ion and crystal growth to form the particles.

In one embodiment, the reducing agent can be ultraviolet-visible light. Upon exposure of the metal ion/carrier composite (for instance in the form of an aqueous suspension) over a period of time (for instance from about 1 hour to about 7 days) the metal nanoparticles can form and the metal can be reduced. When utilizing nanosized titanate carrier, the formation process can be faster as compared to use of larger micron-sized titanate carriers.

Chemical reduction agents can also be utilized to form the metal nanoparticles. By way of example, alcohols such as ethanol can be utilized in one embodiment. In general, reducing agents can include organic compounds including a unit having the structure

—HCR—OR′

wherein
R is H or saturated or unsaturated organic group and
R′ is H or saturated or unsaturated organic group.

According to one embodiment, a suspension of the metal ion/carrier composite particles can be placed in a solution of the reducing agent, and over a period of time (for instance about 30 minutes or more, for example from about 30 minutes to about 1 day), the metal ions are reduced and form nanoparticles.

Multiple reducing agents can be utilized together, which can increase the rate of formation of the nanoparticles. For instance, subjecting a suspension of the particles in a solution of a chemical reducing agent to UV-visible light can increase the rate for formation of the metal nanoparticles and the reduction of the metal.

The morphology of the formed metal nanoparticles can be controlled. For instance, immediately upon formation, metal nanoparticles formed on nanosized monosodium titanate carriers can be generally spherical with a maximum cross sectional dimension of about 10 nanometers. After additional contact with the reducing agent, the metal nanoparticles can form irregular clusters having a larger cross section, for instance from about 20 to about 200 nanometers as a maximum cross sectional dimension. When utilizing sodium titanate nanotubes as a carrier, in contrast, the metal nanoparticles can be spherical with a maximum cross sectional dimension of from about 10 to about 15 nanometers. Thus, the size and shape of the metal nanoparticles can be controlled by variation of the contact time with the reducing agent as well as by variation of the specific titanate carrier.

The composites including the metal nanoparticles can be utilized in a variety of applications. The metal nanoparticles may be utilized in the reduced state, as formed, or alternatively a portion of all of the metal may be oxidized following formation of the metal nanoparticles.

By way of example, the composite materials can be utilized in photocatalytic applications such as in the decomposition of hazardous organics and in solar cells. The composites may be utilized in imaging and detection applications such as SERS. Another application of the composite materials is for use as a conductive electrode in a fuel cell.

In one embodiment, the composite materials may be utilized in medical technologies including diagnostic, imaging, cancer treatment, and wound sterilization/treatment. For instance, in one embodiment the composite materials may be utilized in dental composites and may extend the life of the composites by limiting bacterial-induced corrosion. According to another embodiment, the composite materials may be utilized for delivery of the metal nanoparticles to a biological site for therapeutic purposes, for instance as an anti-inflammatory. For instance, a buffered solution including the composite particles can be introduced into a physiological system for delivery of the metal to a targeted site such as, without limitation, an organ, a joint, a bone, a tissue, a tumor, etc. The composite particles can be delivered according to any delivery method including ingestion, implantation, inhalation, intravenously, etc.

Coatings including the composite materials may beneficially be utilized as a bactericide, for instance in wound dressings, on dental implants, on orthopedic implants, etc.

The present application may be further understood by reference to the following Examples.

### EXAMPLE 1

### Materials

All chemicals were used as received without further purification. Titanium (IV) isopropoxide (TITP) was obtained from either Alfa Aesar (Ward Hill, Mass.) or Sigma-Aldrich (St. Louis, Mo.). HPLC grade isopropyl alcohol (Chromasolv®; absolute, 99.9%) and sodium methoxide in methanol (30 wt %) were obtained from Sigma-Aldrich. Triton® X-100 was obtained from Sigma-Aldrich. Ultrapure water was supplied by a MilliQ Element water purification system.

### Synthesis of Nano-Monosodium Titanate

A first solution—Solution 1—was formed that contained 1.80 cm³ (6 mmol) of TITP, 0.58 cm³ (3 mmol) of ˜30 wt % sodium methoxide, and 7.62 cm³ of isopropanol.

A second solution—Solution 2—was formed that contained 0.24 cm³ (13.5 mmol) of ultrapure water and 9.76 cm³ of isopropanol.

Solutions 1 and 2 were added simultaneously by two syringe pumps to a well-stirred solution of 280 cm³ isopropanol and 0.44 cm³ of a surfactant (Triton® X-100). This step was carried out in a 500 cm³ 2-neck round bottom flask. The rate of addition for solutions 1 and 2 was 0.333 cm³ min⁻¹. After adding Solutions 1 and 2, the flask was sealed and stirred for 24 hours.

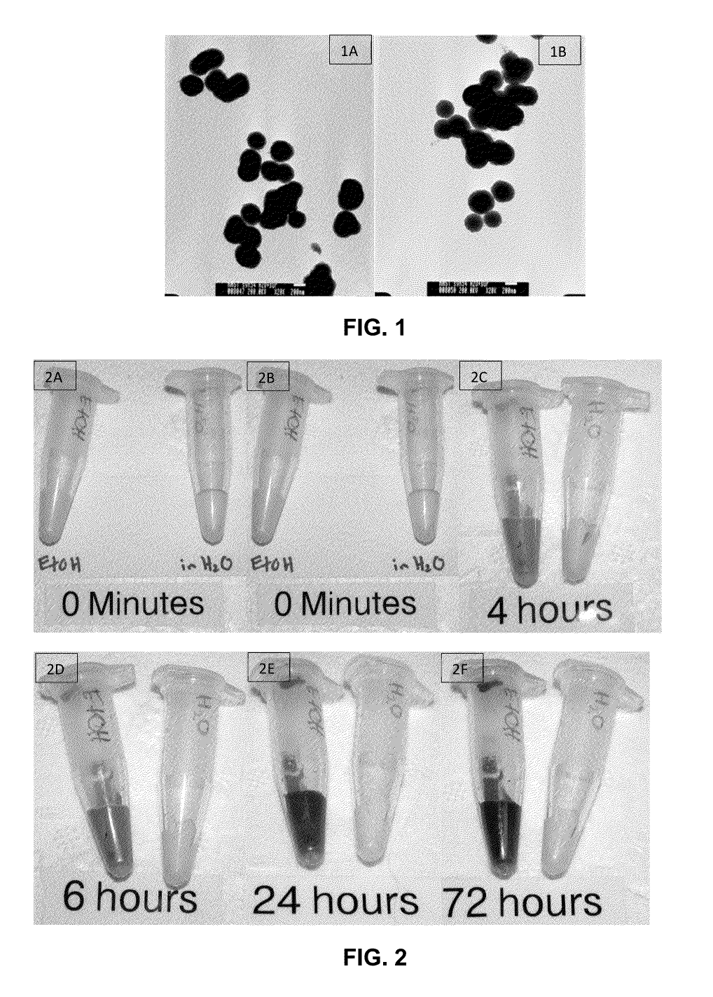

The reaction mixture was heated to 82° C. for 90 minutes followed by purging with nitrogen while maintaining 82° C. As the isopropanol evaporated, ultrapure water was added dropwise. After most of the isopropanol evaporated and the water volume was approximately 50 cm³, the heat was removed. The aqueous slurry was then filtered through 0.1-μm nylon filter paper and the collected product was washed with water to remove any surfactant and any remaining isopropanol. The product was stored as aqueous slurry. FIG. 1A and FIG. 1B present TEM images for nanosized monosodium titanate following formation.

### Au(III) Ion Exchange

For Au ion exchange, 6.0 cm³ of a 30.9 mM solution HAuCl₄3H₂O (pH 3.0) was combined with a suspension of nanosized monosodium titanate, diluted with water to a final volume of 14.4 cm³ and mixed at ambient laboratory temperature for 4-11 days. The solids and solution were separated by filtration and separately analyzed for Au, Ti and Na content by ICP-ES.

ICP-ES analysis of the filtrates after an 11-day contact period indicated loss of Au³⁺ and an increase in Na⁺ concentration. Analysis of the solids showed the presence of Au and reduced Na content. These findings confirm that Au³⁺ exchanged for Na⁺ on the nanosized monosodium titanate. The measured ratio of exchanged Na³⁰ to that of Au³⁺ measured 4.08 for nanosized monosodium titanate compared to a theoretical value of 3.00. The higher ratios suggest additional exchange of Na⁺, which could be by protons since the gold chloride solution is acidic (pH 3.0). Based on solution analyses, the nanosized monosodium titanate removed 93.6% of the dissolved Au³⁺. The gold loading in the isolated solids measured 228 mg Au/g Ti.

### Synthesis of Au(0) Nanoparticles

A moist paste of the gold loaded nanosized titanate particles was suspended in ethanol. As control, a second suspension was formed with the particles in water. FIG. 2 illustrates the two suspensions over a 72 hour period, with the ethanol suspension on the left and the water suspension on the right. As can be seen, the particles darken over time as the Au(III) is reduced to Au(0). These samples were stored in the dark between photographing. Similar results were obtained for a suspension subjected to UV-visible light.

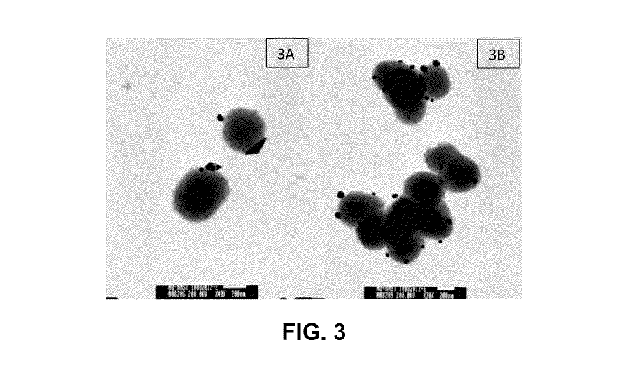

TEM images and elemental mapping of the samples subjected to UV-visible light revealed that before photodecomposition, the Au(III) was uniformly distributed over the nanosized carrier particle and the material was a pale yellow that is stable in air and water. However, upon reduction to the elemental gold form, TEM images reveal the presence of gold nanoparticles having spherical, irregular, trigonal and hexagonal shapes (FIG. 3).

### EXAMPLE 2

### Au(III) Ion Exchange

A sample of micron-sized monosodium titanate (Optima 00-QAB-417) was loaded with Au(III) by contacting an aqueous suspension of the monosodium titanate with an aqueous solution of HAuCl₄ at a Ti:Au mass ratio of 4:1 for 5 days. The Au(III) loaded monosodium titanate was collected by centrifuging, and was washed three times with distilled water to remove any free Au(III). After washing, the final product was redispersed in water and stored in the dark.

### Synthesis of Au(0) Nanoparticles

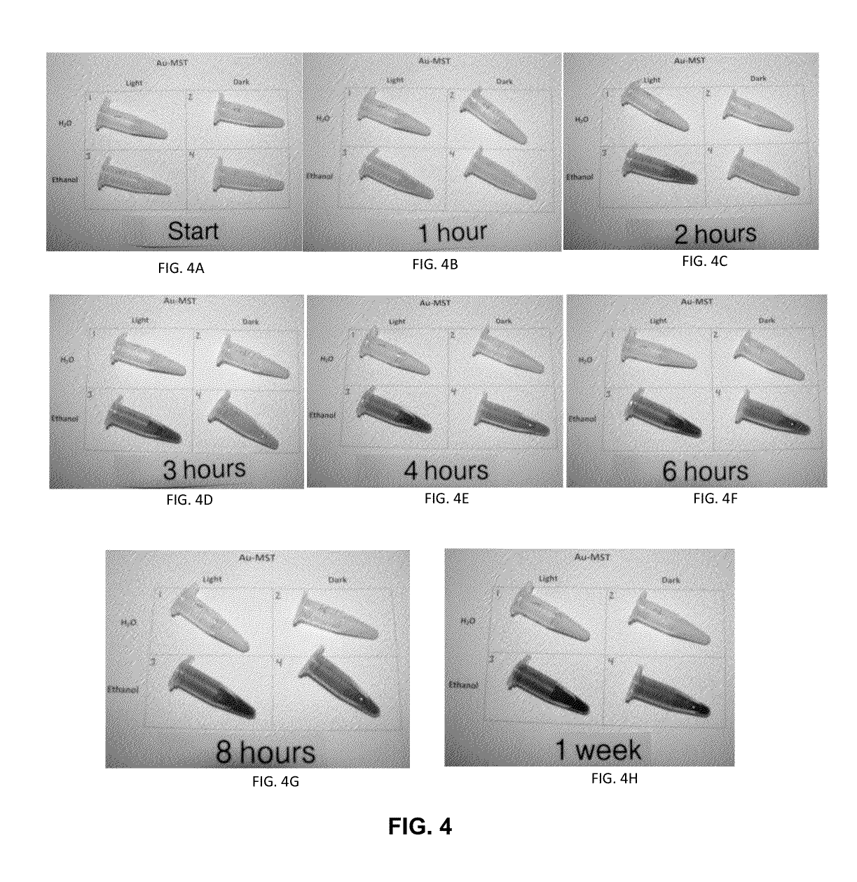

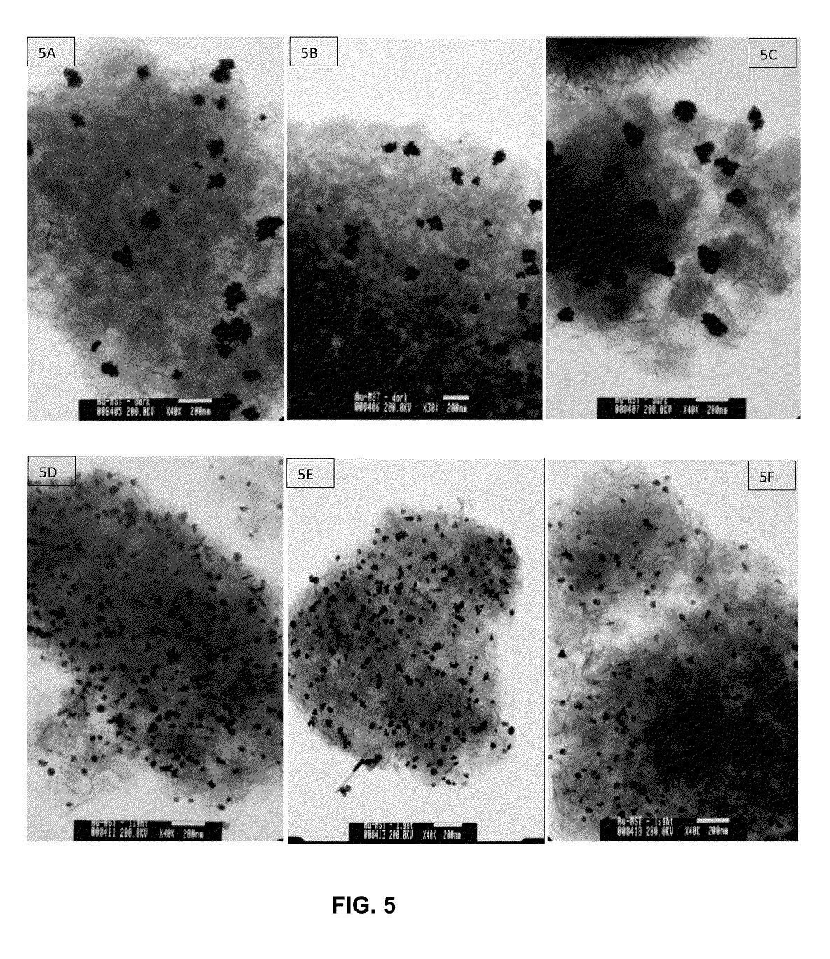

500 μL aliquots of the Au(III) loaded monosodium titanate suspension were placed in four 1.5-mL tubes. Two of the tubes were centrifuged to isolate the solids and these solids were then redispersed in 500 μL of ethanol. The other two remained in aqueous suspension. One of each pair of tubes (ethanol or water) was placed in the dark, while the other was left exposed to light on a benchtop. Photos were taken of all four samples initially, and then after 1, 2, 3, 4, 6, and 8 hours of storage and after 1 week (FIGS. 4A-4H). The samples that were stored in the dark were briefly exposed to the light during each photographing, and were then immediately returned to the dark. As can be seen in the images of FIG. 4, the sample in ethanol stored in the light began to form Au nanoparticles within the first hour, as indicated by the color change. In contrast, the ethanol sample stored in the dark did not show a noticeable color change until 3 hours of contact. The Au(III) loaded monosodium titanate stored in water in the light or dark did not show any evidence of Au nanoparticle formation after 1 week of storage. TEM imaging (FIG. 5) also confirmed the presence of Au nanoparticles on the samples that were stored in ethanol. FIG. 5A, FIG. 5B, and FIG. 5C illustrate those samples that were stored in the dark during the formation and FIG. 5D, FIG. 5E, and FIG. 5F illustrate those samples that were stored in the light during the formation.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

1. A method of forming metal nanoparticles comprising:
depositing metal ions on a titanate carrier, the metal ions having an oxidation state;
following the deposition, exposing the metal ions and the titanate carrier to a reducing agent, wherein upon the exposure nanoparticles of the metal are formed on the titanate carrier, the metal of the nanoparticles being reduced from the oxidation state of the metal ions.

2. The method of claim 1, wherein the titanate carrier is a nanosized titanate carrier.

3. The method of claim 1, wherein the titanate carrier is a micron-sized titanate carrier.

4. The method of claim 1, wherein the metal ions are deposited according to a chemical deposition process.

5. The method of claim 4, wherein the chemical deposition process is an ion exchange process.

6. The method of claim 1, wherein the titanate carrier is monosodium titanate.

7. The method of claim 1, wherein the titanate carrier is sodium peroxotitanate.

8. The method of claim 1, wherein the titanate carrier is sodium titanium oxide nanoparticles.

9. The method of claim 1, wherein the reducing agent comprises an alcohol.

10. The method of claim 9, wherein the alcohol is ethanol.

11. The method of claim 1, wherein the reducing agent comprises ultraviolet-visible light.

12. The method of claim 1, wherein the metal is a transition metal.

13. The method of claim 12, wherein the metal is a metal of the platinum group.

14. The method of claim 13, wherein the metal is gold.

15. The method of claim 1, wherein the deposition is carried out at ambient temperature and pressure.

16. The method of claim 1, wherein the step of exposing the metal ions and titanate carrier to a reducing agent is carried out at ambient temperature and pressure.

17. A composite comprising a titanate carrier and metal nanoparticles adhered to the titanate carrier.

18. The composite of claim 17, wherein the titanate carrier is monosodium titanate.

19. The composite of claim 17, wherein the nanosized titanate carrier is sodium peroxotitanate.

20. The composite of claim 17, wherein the titanate carrier comprises nanosized titanate particles.

21. The composite of claim 17, wherein the titanate carrier comprises micron-sized titanate particles.

22. The composite of claim 17, wherein the titanate carrier comprises sodium titanium oxide nanoparticles.

23. The composite of claim 17, wherein the metal is a transition metal.

24. The composite of claim 23, wherein the metal is a metal of the platinum group.

25. The composite of claim 24, wherein the metal is gold.

26. The composite of claim 17, wherein the composite is free of any organic surfactant.
